(19) United States
(12) Patent Application Publication
(10) Pub. No.: US 2008/0269728 A1
(43) Pub. Date: Oct. 30, 2008

(54) Active Lamp Alignment for Fiber Optic Illuminators

(76) Inventors: Mark J. Buczek, Oceanside, CA (US); Michael Papac, Tustin, CA (US); Ronald T. Smith, Newport Coast, CA (US)

Correspondence Address: ALCON, IP LEGAL, TB4-8, 6201 SOUTH FREEWAY, FORT WORTH, TX, 76134, US

(21) Appl. No.: 11/739,227

(22) Filed: Apr. 24, 2007

(51) Int. Cl. A61B 1/07

(52) U.S. Cl. 606/4; 600/178

(57) ABSTRACT

An assembly for use in an ophthalmic endoilluminator includes a precision lamp assembly, an actuator, and a controller. The precision lamp assembly has a housing and a lamp holder for holding a lamp. The actuator is connected to the precision lamp assembly and is configured to move the precision lamp assembly. The controller controls the operation of the actuator. The controller directs the actuator to move the precision lamp assembly over time to compensate for hot spot movement of the lamp.

### BACKGROUND OF THE INVENTION

The present invention relates to an illuminator for use in ophthalmic surgery and more particularly to ophthalmic illuminator utilizing active lamp alignment to produce a light suitable for illuminating the inside of the eye.

Anatomically, the eye is divided into two distinct parts—the anterior segment and the posterior segment. The anterior segment includes the lens and extends from the outermost layer of the cornea (the corneal endothelium) to the posterior of the lens capsule. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. The vitreous body becomes more fluid with age in a process known as syneresis. Syneresis results in shrinkage of the vitreous body, which can exert pressure or traction on its normal attachment sites. If enough traction is applied, the vitreous body may pull itself from its retinal attachment and create a retinal tear or hole.

Various surgical procedures, called vitreo-retinal procedures, are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

A surgeon performs vitreo-retinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

During such surgical procedures, proper illumination of the inside of the eye is important. Typically, a thin optical fiber is inserted into the eye to provide the illumination. A light source, such as a metal halide lamp, a halogen lamp, a xenon lamp, or a mercury vapor lamp, is often used to produce the light carried by the optical fiber into the eye. The light passes through several optical elements (typically lenses, mirrors, and attenuators) and is launched at the optical fiber that carries the light into the eye. The quality of the illumination is dependent on several factors including the light source.

A xenon lamp used in an ophthalmic illumination system typically has a relatively small arc (e.g., about 0.8 mm gap width for an Osram/Sylvania® 75 W xenon bulb at zero hours operating time). Optics within the illumination system are used to focus an image of the arc onto the optical fiber and the xenon bulb must be precisely aligned to ensure that an optimum amount of light is coupled into the optical fiber, and hence an optimum luminous flux emerges from the fiber. The optical fiber core diameter is selected to be large enough that the arc image will fit within the fiber core area. However, as the xenon bulb ages, the bulb cathode degrades and moves away from the bulb anode. As the cathode degrades, the arc grows in size, decreases in peak luminance and also moves away from the anode.

The xenon bulb is positioned so that the arc image will fall on the optical fiber core entrance surface. In prior art illumination systems, the xenon bulb is positioned such that maximum fiber throughput is achieved at zero hours of operation (i.e., beginning of life of the xenon bulb). However, the arc can move (due to cathode degradation) in excess of about 250 microns during the first 200 hours of operation in a typical illumination system. Therefore, if the xenon bulb is aligned for maximum fiber throughput at zero hours, the arc movement (which can result in much of the arc image moving outside of the fiber core area) combined with the decrease in arc peak luminance can result in an appreciable drop in fiber throughput, and hence in an appreciable drop in illumination at the surgical site.

One way of solving this problem in prior art ophthalmic illumination systems is to increase the diameter of the optical fiber core. However, increasing the diameter of the optical fiber has several disadvantages. The increased fiber diameter results in a stiffer optical fiber, which is not as easy to manipulate in an operating environment. A larger diameter fiber is more expensive because more fiber material is used per unit length of optical fiber. A larger diameter fiber may be greater than that allowed by size requirements on the probe inserted into the eye. If the optical fiber tapers to a smaller diameter downstream from its proximal end, transmittance of light through the fiber is inversely dependent on the taper ratio—the ratio between the fiber proximal diameter and distal diameter. Therefore, for a fixed distal fiber diameter, an increase in proximal fiber diameter will result in a reduction in light transmittance. Therefore, for a fixed distal fiber, even though an increase in proximal diameter may result in more light coupled into the fiber, most if not all of this extra light may not reach the distal end of the fiber due to decreased fiber transmittance.

Therefore, a need exists for a system for enhancing the useful lifetime of an ophthalmic illumination system that can reduce or eliminate the problems of prior art ophthalmic illumination systems discussed above.

### SUMMARY OF THE INVENTION

In one embodiment consistent with the principles of the present invention, the present invention is an ophthalmic endoilluminator comprising a light source, a precision lamp assembly for holding the light source, an actuator for moving the precision lamp assembly, a controller for controlling the operation of the actuator, a collimating lens for collimating light produced by the light source, a condensing lens for focusing the light, and an optical fiber for carrying the focused light into an eye. The actuator moves the precision lamp assembly over time to compensate for movement of a hot spot of the light source.

In another embodiment consistent with the principles of the present invention, the present invention is an assembly for use in an ophthalmic endoilluminator including a precision lamp assembly, an actuator, and a controller. The precision lamp assembly has a housing and a lamp holder for holding a lamp. The actuator is connected to the precision lamp assembly and is configured to move the precision lamp assembly. The controller controls the operation of the actuator. The controller directs the actuator to move the precision lamp assembly over time to compensate for hot spot (or, arc) movement of the lamp.

In another embodiment consistent with the principles of the present invention, the present invention is an ophthalmic endoilluminator. The ophthalmic endoilluminator has a light source, a precision lamp assembly for holding the light source, an actuator for precisely moving the lamp and/or lamp assembly, a controller for controlling the operation of the actuator, an optional reflector for reflecting light from the light source, a collimating lens for collimating the light produced by the light source, a filter for filtering the collimated light, an attenuator for attenuating the filtered light, a condensing lens for focusing the attenuated light, and an optical fiber for carrying the focused light into an eye. The controller directs the actuator to move the precision lamp assembly over time to compensate for movement of the hot spot of the light source.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is an unfolded view of an ophthalmic endoilluminator according to an embodiment of the present invention.

FIG. 2 is a view of an ophthalmic endoilluminator in a sigma configuration according to an embodiment of the present invention.

FIG. 3 is a graph depicting hot spot movement over time of a typical xenon lamp.

FIGS. 4A-4C are exploded views of the location of a hot spot of a xenon lamp with respect to a small diameter optical fiber as the xenon lamp ages.

FIGS. 5A-5C are exploded views of the anode and cathode of a typical xenon lamp as it ages.

FIG. 6A is a front view of a precision lamp assembly according to an embodiment of the present invention.

FIG. 6B is a perspective view of a precision lamp assembly according to an embodiment of the present invention.

FIG. 7A is a front view of a precision lamp assembly according to an embodiment of the present invention.

FIGS. 7B and 7C are perspective views of a precision lamp assembly according to an embodiment of the present invention.

FIG. 8 is a block diagram of a precision lamp assembly system according to an embodiment of the present invention.

FIG. 9 is a view of an endoilluminator probe as used in an eye according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

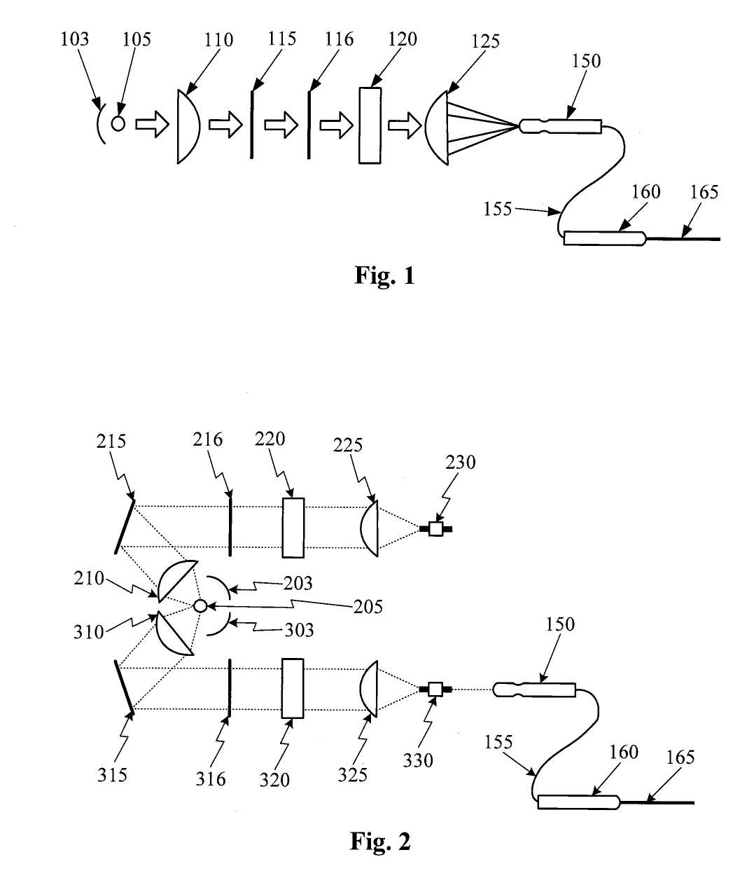

FIG. 1 is an unfolded view of an ophthalmic endoilluminator according to an embodiment of the present invention. In FIG. 1, the endoilluminator includes optional reflector 103, light source 105, collimating lens 110, optional cold mirror 115, optional hot mirror 116, attenuator 120, condensing lens 125, connector 150, optical fiber 155, hand piece 160, and probe 165.

The light from light source 105 is reflected by optional reflector 103 and collimated by collimating lens 110. The collimated light is reflected and filtered by optional cold mirror 115 and/or optional hot mirror 116. The resulting beam is attenuated by attenuator 120 and focused by condensing lens 125. The focused beam is directed through connector 150 and optical fiber 155 to probe 165 where it illuminates the inside of the eye.

Light source 105 is typically a lamp, such as a xenon lamp. Light source 105 is operated at or near full power to produce a relatively stable and constant light output. In one embodiment of the present invention, light source 105 is a xenon lamp with an arc length of about 0.8 mm, such as a 75 watt xenon lamp manufactured by Osram/Sylvania®.

Optional reflector 103 is a spherical or aspherical optional reflector designed to reflect the light emitted by light source 105 toward collimating lens 110. When light source 105 is a xenon lamp, light is emitted from it in all directions around the lamp surface. The light that is emitted from the side of the lamp opposite the collimating lens is reflected by optional reflector 103 so that it passes through collimating lens. In other words, optional reflector 103 serves to direct a greater portion of the light emitted by light source 105 toward the collimating lens. Using optional reflector 103 increases the light directed at collimating lens 110 by about 25%-40%.

Collimating lens 110 is configured to collimate the light produced by light source 105. As is commonly known, collimation of light involves lining up light rays. Collimated light is light whose rays are parallel with a planar wave front.

Optional cold mirror 115 is a dichroic optional reflector that reflects visible wavelength light and only transmits infrared and ultraviolet light to produce a beam filtered of harmful infrared and ultraviolet rays. Optional hot mirror 116 reflects long wavelength infrared light and short wavelength ultraviolet light while transmitting visible light. The eye's natural lens filters the light that enters the eye. In particular, the natural lens absorbs blue and ultraviolet light which can damage the retina. Providing light of the proper range of visible light wavelengths while filtering out harmful short and long wavelengths can greatly reduce the risk of damage to the retina through aphakic hazard, blue light photochemical retinal damage and infrared heating damage, and similar light toxicity hazards. Typically, a light in the range of about 430 to 700 nanometers is preferable for reducing the risks of these hazards. Optional cold mirror 115 and optional hot mirror 116 are selected to allow light of a suitable wavelength to be emitted into an eye. Other filters and/or dichroic beam splitters may also be employed to produce a light in this suitable wavelength range. For example, holographic mirrors may also be used to filter light.

Attenuator 120 attenuates or decreases the intensity of the light beam. Any number of different attenuators may be used. For example, mechanical louvers, camera variable aperture mechanisms, or neutral density filters may be used. A variable-wedge rotating disk attenuator may also be used.

Condensing lens 125 focuses the attenuated light beam so that it can be launched into a small diameter optical fiber. Condensing lens 125 is a lens of suitable configuration for the system. Condensing lens 125 is typically designed so that the resulting focused beam of light can be suitably launched into and transmitted by an optical fiber. As is commonly known, a condensing lens may be a biconvex or plano-convex spherical or aspheric lens. In a plano-convex aspheric lens, one surface is planar and the other surface is convex with a precise aspheric surface in order to focus the light to a minimum diameter spot.

The endoilluminator that is handled by the ophthalmic surgeon includes connector 150, optical fiber 155, hand piece 160, and probe 165. Connector 150 is designed to connect the optical fiber 155 to a main console (not shown) containing light source 105. Connector 150 properly aligns optical fiber 155 with the beam of light that is to be transmitted into the eye. Optical fiber 155 is typically a small diameter fiber that may or may not be tapered. Hand piece 160 is held by the surgeon and allows for the manipulation of probe 165 in the eye. Probe 165 is inserted into the eye and carries optical fiber 155 which terminates at the end of probe 165. Probe 165 thus provides illumination from optical fiber 155 in the eye.

FIG. 2 is a view of an ophthalmic endoilluminator in a sigma configuration according to an embodiment of the present invention. In FIG. 2, the endoilluminator includes optional reflectors 203, 303, light source 205, collimating lenses 210, 310, optional cold mirrors 215, 315, optional hot mirrors 216, 316, attenuators 220, 320, condensing lenses 225, 325, ports 230, 330, connector 150, optical fiber 155, hand piece 160, and probe 165.

The light from light source 205 is reflected by optional reflectors 203, 303 and collimated by collimating lens 210, 310, respectively. The collimated light is filtered by optional cold mirrors 215, 315 and/or optional hot mirrors 216, 316. The resulting beams are attenuated by attenuators 220, 320 and focused by condensing lenses 225, 325, respectively. The beam focused by condensing lens 325 is directed through connector 150 and optical fiber 155 to probe 165 where it illuminates the inside of the eye.

Light source 105 is typically a lamp, such as a xenon lamp. Light source 105 is operated at or near full power to produce a relatively stable and constant light output. In one embodiment of the present invention, light source 105 is a xenon lamp with an arc length of about 0.8 mm, such as a 75 watt xenon lamp manufactured by Osram/Sylvania®.

Optional reflectors 203, 303 are spherical or aspherical optional reflectors designed to reflect the light emitted by light source 105 toward collimating lenses 210, 310. When light source 105 is a xenon lamp, light is emitted from it in all directions around the lamp surface. The light that is emitted from the side of the lamp opposite the collimating lenses is reflected by optional reflector 103 so that it passes through collimating lenses. In other words, optional reflectors 203, 303 serve to direct a greater portion of the light emitted by light source 105 toward the collimating lenses.

Collimating lenses 210, 310, like collimating lens 110, are configured to collimate the light produced by light source 205. As is commonly known, collimation of light involves lining up light rays. Collimated light is light whose rays are parallel with a planar wave front.

Optional cold mirrors 215, 315 are dichroic optional reflectors that reflect visible wavelength light and only transmit infrared and ultraviolet light to produce a beam filtered of harmful infrared and ultraviolet rays. Optional hot mirrors 216, 316 reflect long wavelength infrared light and short wavelength ultraviolet light while transmitting visible light. The eye's natural lens filters the light that enters the eye. In particular, the natural lens absorbs blue and ultraviolet light which can damage the retina. Providing light of the proper range of visible light wavelengths while filtering out harmful short and long wavelengths can greatly reduce the risk of damage to the retina through aphakic hazard, blue light photochemical retinal damage and infrared heating damage, and similar light toxicity hazards. Typically, a light in the range of about 430 to 700 nanometers is preferable for reducing the risks of these hazards. Optional cold mirrors 215, 315 and optional hot mirrors 216, 316 are selected to allow light of a suitable wavelength to be emitted into an eye. Other filters and/or dichroic beam splitters may also be employed to produce a light in this suitable wavelength range. For example, holographic mirrors may also be used to filter light.

Attenuators 220, 320 attenuate or decrease the intensity of the light beams. Any number of different attenuators may be used. For example, mechanical louvers, camera variable aperture mechanisms, or neutral density filters may be used. A variable-wedge rotating disk attenuator may also be used.

Condensing lenses 225, 325 focus the attenuated light beams so that they can be launched into small diameter optical fibers. Condensing lenses 225, 325 are lenses of suitable configuration for the system. Condensing lenses 225, 325 are typically designed so that the resulting focused beams of light can be suitably launched into and transmitted by optical fibers. As is commonly known, a condensing lens may be a biconvex or plano-convex spherical or aspheric lens. In a plano-convex aspheric lens, one surface is planar and the other surface is convex with a precise aspheric surface in order to focus the light to a minimum diameter spot.

Ports 230, 330 receive a connector, such as connector 150, of an ophthalmic endoilluminator. Ports 230, 330 provide a connection between a console (not shown) and an endoilluminator that is handled by the ophthalmic surgeon. Ports 230, 330 also serve to align the optical fiber 155 with the beam of light that is to be transmitted into the eye.

The endoilluminator that is handled by the ophthalmic surgeon includes connector 150, optical fiber 155, hand piece 160, and probe 165. Connector 150 is designed to connect the optical fiber 155 to a main console (not shown) containing light source 105. Connector 150 properly aligns optical fiber 155 with the beam of light that is to be transmitted into the eye. Optical fiber 155 is typically a small diameter fiber that may or may not be tapered. Hand piece 160 is held by the surgeon and allows for the manipulation of probe 165 in the eye. Probe 165 is inserted into the eye and carries optical fiber 155 which terminates at the end of probe 165. Probe 165 thus provides illumination from optical fiber 155 in the eye.

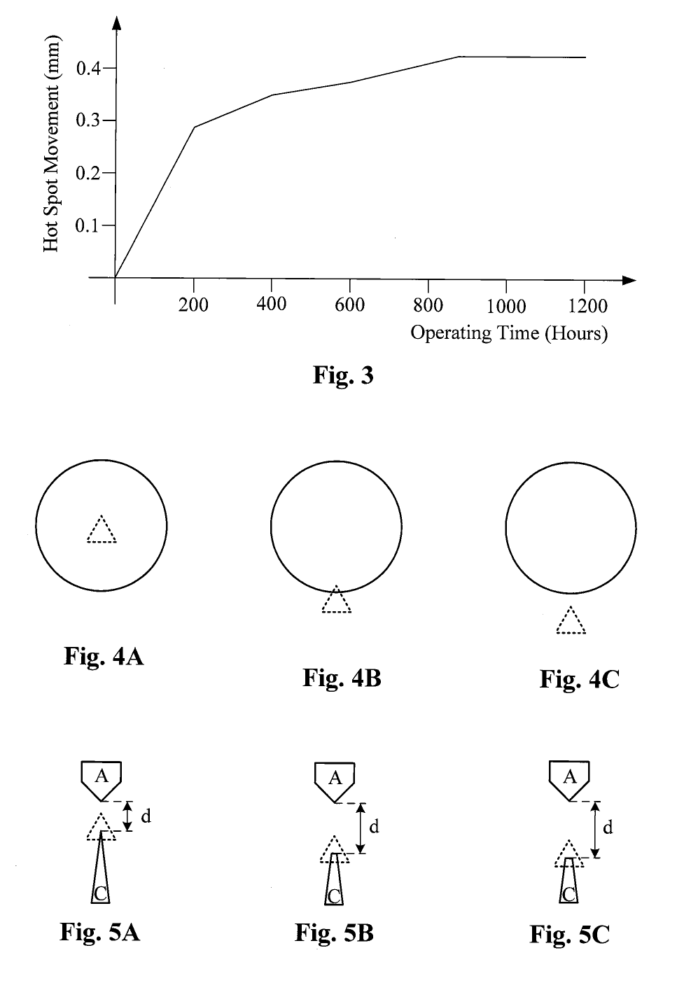

FIG. 3 is a graph depicting hot spot movement over time of a typical xenon lamp. In FIG. 3, time in operating hours is plotted on the x-axis, and hot spot movement in millimeters is plotted on the y-axis. The hot spot of a xenon lamp is the point near the cathode at which light is most highly concentrated. In other words, the hot spot is the area on a luminance plot that exhibits the greatest luminance. As shown, the hot spot moves over time as the cathode erodes. The most movement occurs over the first 200 hours of lamp operating time. In a typical 75 watt xenon lamp, such as that manufactured by OSRAM/Sylvania®, the hot spot moves about 0.3 millimeters over the first 200 hours of lamp operating time.

FIGS. 4A-4C are exploded views of the location of a hot spot of a xenon lamp with respect to a small diameter optical fiber as the xenon lamp ages. In FIGS. 4A-4C, the circles represent a cross section of a 25 gauge optical fiber. A 25 gauge optical fiber has a diameter of about 0.455 millimeters. In FIG. 4A, the location of the hot spot at zero operating hours is represented by the triangle. Likewise, in FIG. 4B, the location of the hot spot at 200 operating hours is represented by the triangle, and in FIG. 4C, the location of the hot spot at 800 operating hours is represented by the triangle. Over time, the hot spot location moves outside the diameter of a 25 gauge optical fiber.

This is also shown in FIGS. 5A-5C, in which the hot spot is depicted by the triangle. FIGS. 5A-5C are exploded views of the anode and cathode of a typical xenon lamp as it ages. The hot spot is located in an area around the tip of the cathode (denoted by the letter “C”). As the cathode, C, erodes over time (as shown in FIGS. 5B and 5C), the location of the hot spot moves. The distance “d” between the anode “A” and the cathode “C” increases as the cathode “C” erodes. In FIG. 5A, the location of the hot spot at zero operating hours is represented by the triangle. Likewise, in FIG. 4B, the location of the hot spot at 200 operating hours is represented by the triangle, and in FIG. 4C, the location of the hot spot at 800 operating hours is represented by the triangle.

As seen in FIG. 3, FIGS. 4A-4C and FIGS. 5A-5C, the hot spot moves over time. The most significant movement occurs in the first 200 hours of operating time. The hot spot, where the greatest luminance occurs, moves as the cathode “C” erodes. When a 25 gauge optical fiber is used, as depicted in FIGS. 4A-4C, the hot spot moves outside the fiber diameter. In such a case, much of the light is lost. When the hot spot moves outside the fiber diameter, the light transmitted by the fiber greatly decreases.

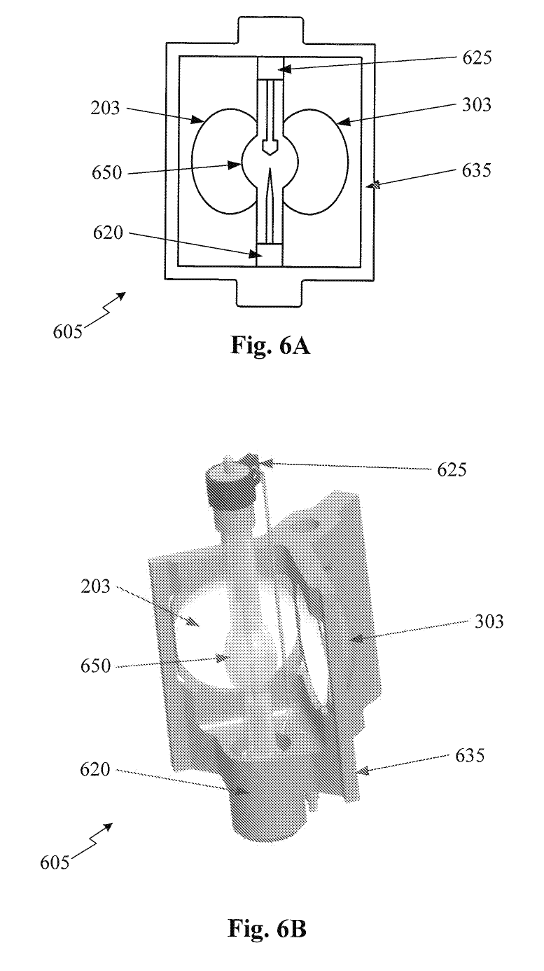

To solve this problem, the hot spot can be moved so that it remains centered on the optical fiber over time. FIG. 6A is a front view of a precision lamp assembly according to an embodiment of the present invention. The precision lamp assembly 605 of FIG. 6A permits the lamp to be moved a precise distance over a period time. As the hot spot moves, the precision lamp assembly 605 also moves to keep the hot spot centered on the optical fiber to maintain a relatively high light output over time. Precision lamp assembly 605 also allows for the use of smaller diameter optical fibers, such as 25 gauge optical fibers.

Precision lamp assembly 605 includes reflectors 203 and 303, lamp holders 620 and 625, lamp 650, and housing 635. Reflectors 203 and 303 are as described in FIG. 2. Lamp 650 is preferably a xenon lamp. Lamp holders 620 and 625 are designed to hold lamp 650 in a position within housing 635. Lamp holders 620 and 625 are precisely located in housing 635 so that lamp 650, and its hot spot, can be precisely located and moved over time. While shown with two optional reflectors 203 and 303, the precision lamp assembly 605 of FIG. 6 may be configured with a single optional reflector as depicted in FIG. 1 or with no reflectors at all.

FIG. 6B is a perspective view of a precision lamp assembly according to an embodiment of the present invention. Precision lamp assembly 605 includes reflectors 203 and 303, lamp holders 620 and 625, lamp 650, housing 635.

In one embodiment of the present invention, reflectors are not included in precision lamp assembly 605. Instead, reflectors are separate from precision lamp assembly 605. In this embodiment, precision lamp assembly 605 moves over time to correct for hot spot movement of lamp 650. Making the reflectors stationary (and not a part of the precision lamp assembly) means that as precision lamp assembly 605 moves to keep the hot spot in the same location, the reflectors have the same spatial relationship with the hot spot. If the reflectors are incorporated into precision lamp assembly 605, then the hot spot will move with respect to the reflectors. In such a case, the precision lamp assembly 605 cannot fully correct for movement of the reflected image of the hot spot (i.e. the projected image of the hot spot or arc moves with respect to the optics, but the reflected image does not).

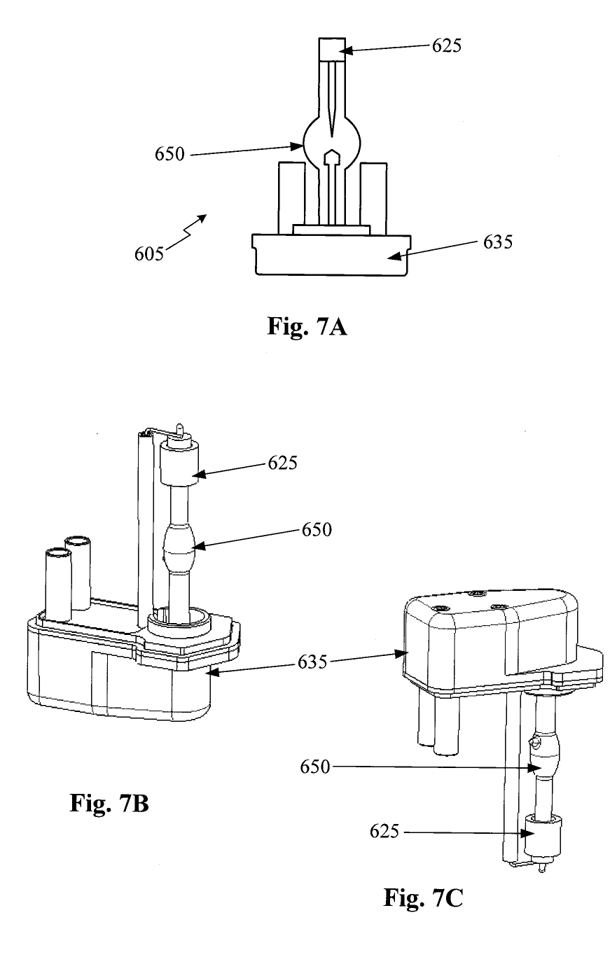

This embodiment is shown in FIG. 7A-7C. FIG. 7A is a front view of a precision lamp assembly without reflectors. Precision lamp assembly 605 includes lamp holder 625, housing 635, and lamp 650. Another lamp holder is located in housing 635 at the bottom end of lamp 650. FIGS. 7B and 7C are perspective views of the precision lamp assembly 605 of FIG. 7A.

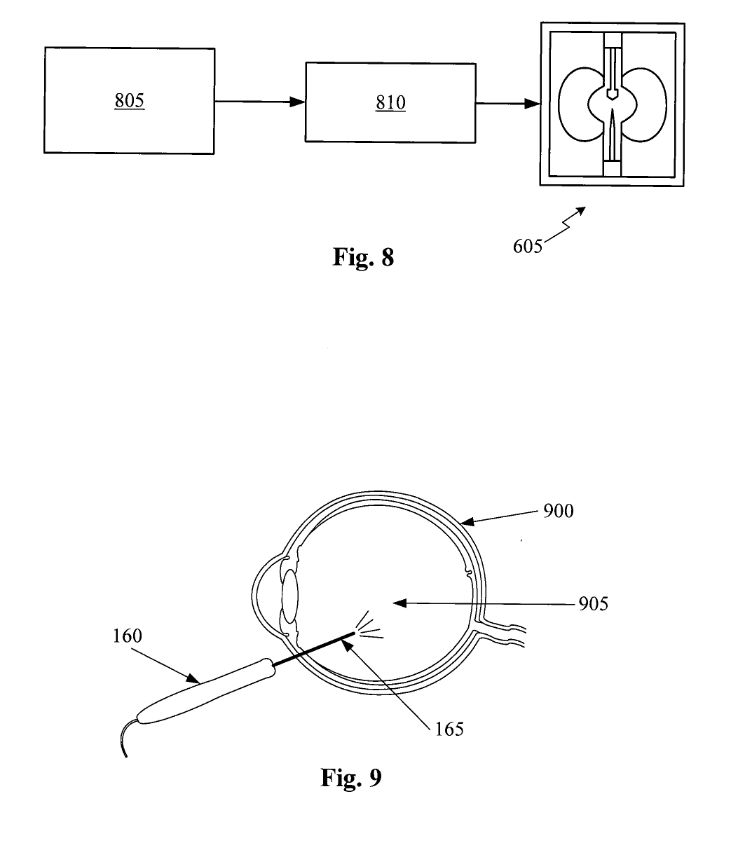

FIG. 8 is a block diagram of a precision lamp assembly system according to an embodiment of the present invention. In FIG. 8, controller 805 interfaces with actuator 810 that precisely moves precision lamp assembly 605.

Controller 805 controls the operation of the actuator 810 and is typically an integrated circuit with power, input, and output pins capable of performing logic functions. In various embodiments, controller 805 is a targeted device controller performing specific control functions targeted to a specific device or component, such as directing the operation of the actuator 810. In other embodiments, controller 805 is a programmable microprocessor. Software loaded into the microprocessor implements the control functions provided by controller 805. Controller 805 may be made of many different components or integrated circuits.

Actuator 810 moves precision lamp assembly 605 to compensate for movement of the hot spot of the xenon lamp over time. Actuator 810 is capable of moving precision lamp assembly 605 small distances, such as tenths or hundredths of a millimeter. In several embodiments of the present invention actuator 810 is a piezoelectric actuator, a precise mechanical translator, or a precision electric motor.

Controller 805 interfaces with a memory (not shown) which may be included on the same or a separate integrated circuit as controller 805 or may be a separate component. The memory contains information about the characteristics of the xenon lamp used in the endoilluminator including information about how the hot spot moves over time. This information is used by controller 805 to control the movement of actuator 810. For example, the hot spot of a typical 75 watt xenon bulb manufactured by OSRAM/Sylvania® moves as shown in FIGS. 3-5. At 200 hours of operating time, controller 805 directs actuator 810 to move the precision lamp assembly 805 approximately 0.3 millimeters. This in turn moves the hot spot of the bulb to the proper centered location on the optical fiber.

In order to properly move precision lamp assembly 650, controller 805 keeps track of the operating time of lamp 205. For example, controller 805 has a counter (not shown) that records the time that the lamp is on or the time that power is applied to the lamp. Controller 805 uses this recorded time to determine the proper location of precision lamp assembly 805. Controller 805 directs actuator 810 to move the lamp incrementally depending on the recorded time and the hot spot movement characteristics of the lamp.

In other embodiments of the present invention, memory (not shown) in controller 805 has information about the hot spot movement over time of several different lamps. Controller 805 identifies the type of lamp inserted in precision lamp assembly 605, for example, by an RFID system or by a user input. The type of lamp may also be designated at the factory when it is installed or at the time the lamp is replaced.

In further embodiments of the present invention, an initial offset is stored in the memory (not shown) with which controller 805 interfaces. This initial offset describes the initial position of the precision lamp assembly 605 in the ophthalmic endoilluminator. The initial offset may be determined at the factory before the endoilluminator is shipped to the customer. In other embodiments, the initial position of the precision lamp assembly is set and calibrated at the factory. A locking mechanism (not shown) keeps the precision lamp assembly in place during use. When the lamp is replaced, the precision lamp assembly is returned to its original position and locked in place by the locking assembly (not shown). In addition, when the lamp is replaced, the lamp operating time counter (not shown) is reset by controller 805. In other words, controller 805 detects when the lamp has been replaced and resets the counter so that the new lamp can be properly positioned by actuator 810 over time.

In another embodiment of the present invention, controller 805 interfaces with a light sensor (not shown). The light sensor (not shown) can be located at any point along the light path of the endoilluminator. The light sensor (not shown) provides feedback to the controller 805 about the intensity of the light. The controller 805 uses this feedback to move precision lamp assembly 605 to provide proper light output. In one embodiment, the light sensor (not shown) is connected to an optical fiber (not shown) that is attached to port 230. The light sensor (not shown) reads the light output at port 230 and provides the information to the controller 805.

FIG. 9 is cross section view of an ophthalmic endoilluminator located in an eye according to an embodiment of the present invention. FIG. 9 depicts hand piece 160 and probe 165 in use. Probe 165 is inserted into eye 900 through an incision in the pars plana region. Probe 165 illuminates the inside or vitreous region 905 of eye 900. In this configuration, probe 165 can be used to illuminate the inside or vitreous region 905 of eye 900 during vitreo-retinal surgery.

From the above, it may be appreciated that the present invention provides an improved system for illuminating the inside of the eye. The present invention provides a light source that can be actively moved to provide a light suitable for illuminating the inside of an eye. A moveable mechanism places the light source at the proper location to compensate for hot spot movement over time. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic endoilluminator comprising:
a light source;
a precision lamp assembly for holding the light source;
an actuator for moving the precision lamp assembly;
a controller for controlling the operation of the actuator;
a collimating lens for collimating light produced by the light source;
a condensing lens for focusing the light; and
an optical fiber for carrying the focused light into an eye;
wherein the actuator moves the precision lamp assembly over time to compensate for movement of a hot spot of the light source.

2. The ophthalmic endoilluminator of claim 1 further comprising:
a reflector for reflecting the light produced by the light source;

3. The ophthalmic endoilluminator of claim 1 further comprising:
a filter for filtering the light exiting the collimating lens;

4. The ophthalmic endoilluminator of claim 3 wherein the filter comprises a cold mirror.

5. The ophthalmic endoilluminator of claim 3 wherein the filter comprises a hot mirror.

6. The ophthalmic endoilluminator of claim 1 further comprising:
an attenuator for attenuating the light.

7. The ophthalmic endoilluminator of claim 1 further comprising:
a connector for aligning the light exiting the condensing lens with the optical fiber;
a hand piece carrying the optical fiber, the hand piece capable of being manipulated in the hand; and
a probe for carrying the optical fiber into the eye.

8. The ophthalmic endoilluminator of claim 7 further comprising:
a port attachable to and detachable from the connector, the port for aligning the light exiting the condensing lens with the optical fiber.

9. The ophthalmic endoilluminator of claim 1 wherein the precision lamp assembly further comprises:
a lamp holder for holding the light source; and
a housing rigidly connected to the lamp holder.

10. The ophthalmic endoilluminator of claim 1 wherein the light source is a xenon lamp.

11. The ophthalmic endoilluminator of claim 1 wherein the controller operates the actuator to move the precision lamp assembly over time to keep the hot spot substantially centered on the optical fiber.

12. The ophthalmic endoilluminator of claim 10 wherein the controller operates the actuator to move the precision lamp assembly over time to compensate for movement of the hot spot caused by erosion of a cathode of the xenon lamp.

13. The ophthalmic endoilluminator of claim 10 further comprising:
a memory for storing values of hot spot movement over time for the xenon lamp, the values used by the controller to move the precision lamp assembly to compensate for hot spot movement.

14. The ophthalmic endoilluminator of claim 1 further comprising:
a light sensor for providing feedback to the controller, the feedback used by the controller to control the operation of the actuator to move the precision lamp assembly.

15. An assembly for use in an ophthalmic endoilluminator comprising:
a precision lamp assembly comprising a housing and a lamp holder for holding a lamp;
an actuator connected to and configured to move the precision lamp assembly; and
a controller for controlling the operation of the actuator;
wherein the controller directs the actuator to move the precision lamp assembly over time to compensate for hot spot movement of the lamp.

16. The assembly of claim 15 further comprising:
a reflector held by the housing, the reflector for reflecting light produced by the lamp.

17. The assembly of claim 15 wherein the lamp holder is configured to hold a xenon lamp.

18. The assembly of claim 15 wherein the controller operates the actuator to move the precision lamp assembly over time to keep the hot spot substantially centered on an optical fiber.

19. The assembly of claim 15 further comprising:
a memory for storing values of hot spot movement over time for a xenon lamp, the values used by the controller to operate the actuator to move the precision lamp assembly to compensate for hot spot movement.

20. An ophthalmic endoilluminator comprising:
a light source;
a precision lamp assembly for holding the light source;
an actuator for moving the precision lamp assembly;
a controller for controlling the operation of the actuator;
a reflector for reflecting light from the light source;
a collimating lens for collimating the light produced by the light source;
a filter for filtering the collimated light;
an attenuator for attenuating the filtered light;
a condensing lens for focusing the attenuated light; and
an optical fiber for carrying the focused light into an eye;
wherein the controller directs the actuator to move the precision lamp assembly over time to compensate for movement of the hot spot of the light source.

21. The ophthalmic endoilluminator of claim 20 further comprising:
a connector for aligning the focused light with the optical fiber;
a hand piece carrying the optical fiber, the hand piece capable of being manipulated in the hand; and
a probe for carrying the optical fiber into the eye.

22. The ophthalmic endoilluminator of claim 21 further comprising:
a port attachable to and detachable from the connector, the port for aligning the focused light with the optical fiber.

23. The ophthalmic endoilluminator of claim 20 wherein the precision lamp assembly further comprises:
a lamp holder for holding the light source; and
a housing connected to the lamp holder.

24. The ophthalmic endoilluminator of claim 20 wherein the light source is a xenon lamp.

25. The ophthalmic endoilluminator of claim 20 wherein the controller operates the actuator to move the precision lamp assembly over time to keep the hot spot substantially centered on the optical fiber.

26. The ophthalmic endoilluminator of claim 24 further comprising:
a memory for storing values of hot spot movement over time for the xenon lamp, the values used by the controller to move the precision lamp assembly to compensate for hot spot movement.
